(19) United States
(12) Patent Application Publication
(10) Pub. No.: US 2017/0165122 A1
(43) Pub. Date: Jun. 15, 2017

(54) CLEANING LIQUID ABSORBING TOOL AND METHOD FOR PRODUCING SAME

(71) Applicant: UNIVERSAL VIEW CO., LTD, Tokyo (JP)

(72) Inventors: Takeshi Ide, Tokyo (JP); Sunao Mikawa, Tokyo (JP); Eiichi Uemura, Hyogo (JP); Yoshio Suzuki, Chiba (JP)

(73) Assignee: Universal View Co., Ltd., Tokyo (JP)

(21) Appl. No.: 15/112,496

(22) PCT Filed: Jan. 19, 2015
(86) PCT No.: PCT/JP2015/051265

§ 371 Date: Jul. 19, 2016

(30) Foreign Application Priority Data
Jan. 20, 2014 (JP) 2014-007875

(51) Int. Cl. A61F 13/36; A61L 15/24; A61M 27/00; A61F 13/15; A61B 90/20; A61F 9/007

(52) U.S. Cl. CPC A61F 13/36; A61B 90/20; A61F 9/007; A61M 27/00; A61F 13/15203; A61F 13/15577; A61L 15/24; A61F 2013/15471

(57) ABSTRACT

A surgical fluid-absorbing instrument to enable absorption of an amount of fluid several times as large as by a conventional surgical sponge system and to substantially decreased the times of changing surgical sponges during surgery. A tip sponge part, a retaining sponge part for absorbing the fluid, provided on a position continued from the tip sponge part, and a tubular handle part in which the tip sponge part is loaded are provided. At least the retaining sponge part is water-absorbing. The water-absorbing sponge may be compressed in one direction and solidified, or the water-absorbing sponge may extended in one direction and swollen.

### TECHNICAL FIELD

The present invention relates to a surgical fluid-absorbing instrument, which is applicable to a surgical sponge for medical application, for absorbing blood occurring in an ophthalmologic surgery, a surgical operation or the like (hereinafter, simply referred to as “surgery”) using a microscope and/or surgical fluid used for washing a relevant wound, and a method of producing the same.

### BACKGROUND

In the past, the blood occurring in the ophthalmologic surgery or the like using the microscope, the surgical fluid used for washing the relevant wound and/or the like have often considerably deteriorated any visibility. Accordingly, the surgery has been operated while these blood and surgical fluid have been frequently removed by any surgical sponge or the like in the surgery. For example, a surgical sponge used in a present ophthalmologic surgery utilizes a nature of water-absorbing sponge provided at a tip of a shaft in which when it contains the fluid, it swells.

FIG. 15A is a plan view of a surgical sponge 500 according to a conventional example for illustrating a configuration example thereof. FIGS. 15B and 15C are sectional views of the surgical sponge 500, taken along arrows X5-X5, for illustrating state examples thereof before and after the use thereof. The surgical sponge 500 shown in FIG. 15A is provided with a tip sponge part 51 and a handle part 54 having a length like an extent of a toothpick. The handle part 54 has a fitting part 52 and a rod part 53. The tip sponge part 51 before the use thereof is compressed, dried and solidified to form a triangle and fitted into the fitting part 52 of the handle part 54 as shown in FIG. 15B.

A shape of the surgical sponge 500 after the use thereof becomes a three-dimensional structure having thickness, in which it absorbs any fluid and swells so as to be changed from the compressed, dried and solidified state shown in FIG. 15B to a dustpan-like state shown in FIG. 15C. An operator deals with the surgical sponge 500 so that, for example, it slides along a surface of an eyeball to absorb the surgical fluid.

As a method of using the surgical sponge 500, it does not absorb the water but the surgical sponge softened by absorbing the fluid may be also used instead of a brush, which forms a flap on a surface of a cornea using a part thereof at a refractive surgery and, when it returns to the cornea transpired by a laser, stretches the returned cornea flap so as to prevent a surface thereof from creasing.

Moreover, as to the surgical fluid-absorbing instrument using the water-absorbing sponge, a patent document 1 discloses an apparatus which absorbs water for a laser-assisted in situ keratomileusis surgery. This apparatus which absorbs water has a handle portion and a sickle-shaped portion and a sponge portion is coupled to the sickle-shaped portion.

A patent document 2 discloses a secretion absorber for medical use. This absorber is provided with a hollow shaft portion and a water-absorbing member which is continuous at a tip and in the shaft. The integrated water-absorbing member absorbs a large amount of secretion, blood and/or the like.

A patent document 3 discloses a suction tool for surgical operation. This suction tool is provided with a hollow soft thin film member, a moisture absorbing member loaded in the thin film member, a tip of which is exposed, and a vacuum apparatus which is connected to a rear end of the thin film member. This suction tool absorbs body fluids, which have been absorbed by the absorbing member attaining any profound relevant wound in a surgical field, by operating the vacuum apparatus.

### DOCUMENT FOR PRIOR ART

### Patent Documents

Patent Document 1: U.S. Pat. No. 6,514,223
Patent Document 2: Japanese Patent Application Publication No. H10-108832
Patent Document 3: Japanese Patent Application Publication No. H06-285155

### SUMMARY OF THE INVENTION

### Problems to be Solved by the Invention

By the way, in the patent document 1 and the surgical sponge 500 and the like according to the conventional example, there have been problems as follows:

(i) An amount of absorbed water in the surgical sponge 500 is dependent on volume of the tip sponge part 51 at its swelling time and its water absorption rate is about 0.2 ml through 0.3 ml. Therefore, it can absorb a bit amount of blood and/or fluid but an amount of absorbed water by one surgical sponge 500 may be not enough in water absorption such as washing of wound after the surgery. Accordingly, in an ophthalmologic surgery, there is a problem such that many surgical sponges 500 are required. The similar problem is applied to the apparatus which absorbs water described in the patent document 1.

(ii) According to the apparatus which absorbs water described in the patent document 1, the secretion absorber for medical use disclosed in the patent document 2, the surgical sponge 500 and the like, it is a true state of affairs to be unable to understand how degree the tip sponge part 51 in now use absorbs the water and how degree it could absorb the water thereafter. The amount of absorbed water in the surgical sponge 500 in now use reaches a limit thereof so that only a determination method therefor is a determination in a condition where no fluid is contained therein. Therefore, there is any inconvenience such that the surgical sponges 500 have to be changed even during the surgery.

(iii) In this connection, in order to solve the above problems, it is conceivable to move the fluid absorbed by the surgical sponge 500 to another place using a pump or the like (see the patent document 3), but a hose, a tube or the like for moving the fluid from the surgical sponge 500 is required. Accordingly, in a case where the field of surgery is a surface of relevant wound, behavior and/or functionality of the surgical sponge 500 are considerably deteriorated. Therefore, this is inconvenient as the surgical fluid-absorbing instrument which is compatible for both of a corneal surgery in which the field of surgery is the surface of relevant wound and a coronary artery surgery or the like in which the field of surgery is the relevant profound wound, so that there has been a problem of being short of a versatility.

Accordingly, this invention has solved these problems and has an object to provide a surgical fluid-absorbing instrument, which can absorb an amount of fluid several times as large as that of the surgical sponge with the conventional system and can substantially decrease the changing times of the surgical sponges during the surgery, and a method of producing the same.

### Means for Solving the Problems

In order to solve the above problems, a surgical fluid-absorbing instrument claimed in claim 1 contains a first fluid-absorbing part that absorbs fluid, a second fluid-absorbing part that absorbs the fluid, the second fluid-absorbing part being provided on a portion continued from the first fluid-absorbing part, and a retaining member in which the second fluid-absorbing part is loaded, wherein at least the second fluid-absorbing part is composed of water-absorbing sponge, and two modes of a state in which the water-absorbing sponge is compressed on one direction and solidified and a state in which the water-absorbing sponge is extended on one direction and swollen are born.

According to the surgical fluid-absorbing instrument claimed in Claim 1, the fluid absorbed from the first fluid-absorbing part can be stored in the retaining member so that it is possible to absorb the amount of the fluid several times as large as that of the surgical fluid-absorbing instrument with the conventional system.

In the surgical fluid-absorbing instrument claimed in Claim 2, according to Claim 1, the surgical fluid-absorbing instrument is composed of a water-absorbing sponge in which the first and second fluid-absorbing parts are integrated.

In the surgical fluid-absorbing instrument claimed in Claim 3, according to Claim 2, the first fluid-absorbing part has a compressed and solidified shape in which one of the water-absorbing sponge is compressed on one direction, and the second fluid-absorbing part has a compressed and solidified shape in which the water-absorbing sponge is compressed on a direction that is perpendicular to the compressed direction of the water-absorbing sponge in the first fluid-absorbing part.

In the surgical fluid-absorbing instrument claimed in Claim 4, according to Claim 1, the first fluid-absorbing part is composed of a first fluid-absorbing member in which one of the water-absorbing sponge is compressed on one direction, and the second fluid-absorbing part is composed of a second fluid-absorbing member in which the other water-absorbing sponge is compressed on a direction that is perpendicular to the compressed direction of the water-absorbing sponge in the first fluid-absorbing part.

In the surgical fluid-absorbing instrument claimed in Claim 5, according to Claim 4, the first and second fluid-absorbing parts are configured to be connected to each other with a compressed direction of the first fluid-absorbing part and a compressed direction of the second fluid-absorbing part being perpendicular to each other.

In the surgical fluid-absorbing instrument claimed in Claim 6, according to Claim 1, the retaining member is composed of visible material through which an internal portion thereof is transparently seen.

In the surgical fluid-absorbing instrument claimed in Claim 7, according to Claim 6, the visible material is configured to be a flexible tube having at least any one quality of reversibility and flexibility.

In the surgical fluid-absorbing instrument claimed in Claim 8, according to Claim 6, one water-absorbing sponge or more is loaded into the visible material in their compressed and solidified state, with their swelling directions being aligned.

In the surgical fluid-absorbing instrument claimed in Claim 9, according to Claim 6, a drain port is provided at a rear end of the visible material.

In the surgical fluid-absorbing instrument claimed in Claim 10, according to Claim 9, the visible material has a bellows structure which is expandable to at least a direction that is perpendicular to a longitudinal direction of the visible material.

In the surgical fluid-absorbing instrument claimed in Claim 11, according to Claim 9, a long hose is connected to the drain port of the visible material.

In the surgical fluid-absorbing instrument claimed in Claim 12, according to Claim 11, a drainage device is connected to a rear end of the hose.

In the surgical fluid-absorbing instrument claimed in Claim 13, according to Claim 1, the first fluid-absorbing part is composed of a first fluid-absorbing member in which the isotropic water-absorbing sponge is processed to have a desired shape.

In the surgical fluid-absorbing instrument claimed in Claim 14, according to Claim 1, a window is provided in the water-absorbing sponge constituting the first fluid-absorbing part.

A method of producing a surgical fluid-absorbing instrument claimed in Claim 15 contains a step of processing a water-absorbing sponge to have a desired dimension and forming a tip fluid-absorbing part, a step of compressing and solidifying a water-absorbing sponge which is continued to the tip fluid-absorbing part and forming a retaining fluid-absorbing part which is able to be swollen to one direction, and a step of loading the retaining fluid-absorbing part into a tubular retaining member.

### Effects of the Invention

According to the surgical fluid-absorbing instrument claimed in claim 1, it is configured so that the second fluid-absorbing part provided on a portion continued from the first fluid-absorbing part is loaded in the retaining member to absorb the fluid. Further, the second fluid-absorbing part containing the fluid can be swollen on one direction of the retaining member and the fluid can be smoothly moved to one direction of the retaining member.

This configuration enables the fluid absorbed from the first fluid-absorbing part to be stored in the retaining member so that it is possible to absorb the amount of the fluid several times as large as that of the surgical fluid-absorbing instrument with the conventional system. Accordingly, it is possible to decrease the changing times of the surgical sponge substantially during the surgery. Of course, in a surgery using a microscope, an effect such that an operator does not look away from a surgical field largely may be also attained.

According to the method of producing a surgical fluid-absorbing instrument claimed in claim 15, it is configured so that the compressed and solidified retaining fluid-absorbing part, which is able to be swollen to one direction, is attached to the retaining member so as to be loaded.

This configuration enables the surgical fluid-absorbing instrument which can store the fluid absorbed from the tip fluid-absorbing part in the retaining member to be reproducibly produced. Accordingly, it is possible to provide the surgical sponge which can absorb a large amount of blood occurring within an ophthalmologic surgery or the like using a microscope and/or surgical fluid used for washing a relevant wound.

### BRIEF DESCRIPTION OF DRAWINGS

FIG. 1 is a perspective view of a surgical sponge 100 as a first embodiment according to the invention for illustrating a configuration example thereof.

FIG. 2 is a perspective view of the surgical sponge 100 for illustrating a functional example thereof.

FIG. 3A is a diagram illustrating a formation example of the surgical sponge 100.

FIG. 3B is a diagram illustrating a formation example (Part One) of the surgical sponge 100.

FIG. 3C is a diagram illustrating the formation example (Part Two) of the surgical sponge 100.

FIG. 3D is a diagram illustrating the formation example (Part Three) of the surgical sponge 100.

FIG. 4A is a plan view of the surgical sponge 100 for illustrating a state example before the use thereof.

FIG. 4B is a sectional view of the surgical sponge 100, taken along arrows X1-X1 shown in FIG. 4A, for illustrating a state example before the use thereof.

FIG. 4C is a sectional view of the surgical sponge 100, taken along arrows X1-X1 shown in FIG. 4A, for illustrating a state example after the use thereof.

FIG. 5A is a sectional view of the surgical sponge 100 for illustrating a checking example (Part One) of an amount of absorbed water therein.

FIG. 5B is a sectional view of the surgical sponge 100 for illustrating the checking example (Part Two) of the amount of absorbed water therein.

FIG. 5C is a sectional view of the surgical sponge 100 for illustrating the checking example (Part Three) of the amount of absorbed water therein.

FIG. 5D is a sectional view of the surgical sponge 100 for illustrating the checking example (Part Four) of the amount of absorbed water therein.

FIG. 6A is a sectional view of the surgical sponge 100 for illustrating a drain example of fluid therefrom.

FIG. 6B is a sectional view of the surgical sponge 100 for illustrating another drain example of fluid therefrom.

FIG. 7A is a sectional view of a surgical sponge 200 as a variation example for illustrating a configuration example before the use thereof.

FIG. 7B is a sectional view of the surgical sponge 200, taken along arrows X2-X2 shown in FIG. 7A, for illustrating a state example after the use thereof.

FIG. 8A is a sectional view of a surgical sponge 300 as another variation example for illustrating a configuration example before the use thereof.

FIG. 8B is a sectional view of the surgical sponge 300, taken along arrows X3-X3 shown in FIG. 8A, for illustrating a state example after the use thereof.

FIG. 9 is a perspective view of a surgical sponge 400 as a second embodiment for illustrating an assembled configuration example thereof.

FIG. 10 is an exploded perspective view of a handle portion 42 for illustrating a configuration example thereof.

FIG. 11A is a sectional view of the surgical sponge 400, taken along arrows X4-X4 shown in FIG. 9, for illustrating a state example before the use thereof.

FIG. 11B is a sectional view of the surgical sponge 400, taken along arrows X4-X4 shown in FIG. 9, for illustrating a state example after the use thereof.

FIG. 11C is a sectional view of the surgical sponge 400, taken along arrows X4-X4 shown in FIG. 9, for illustrating a drain example of the fluid therein.

FIG. 12 is a perspective view of a surgical sponge 401 as a variation example for illustrating a configuration example thereof.

FIG. 13 is a perspective view of a surgical sponge 402 as another variation example for illustrating a configuration example thereof.

FIG. 14 is a perspective view of a surgical sponge 403 as still another variation example for illustrating a configuration example thereof.

FIG. 15A is a plan view of a surgical sponge 500 according to a conventional example for illustrating a configuration example thereof.

FIG. 15B is a sectional view of the surgical sponge 500, taken along arrows X5-X5 shown in FIG. 15A, before the use thereof.

FIG. 15C is a sectional view of the surgical sponge 500, taken along arrows X5-X5 shown in FIG. 15A, after the use thereof.

### EMBODIMENT FOR CARRYING OUT THE INVENTION

### First Embodiment

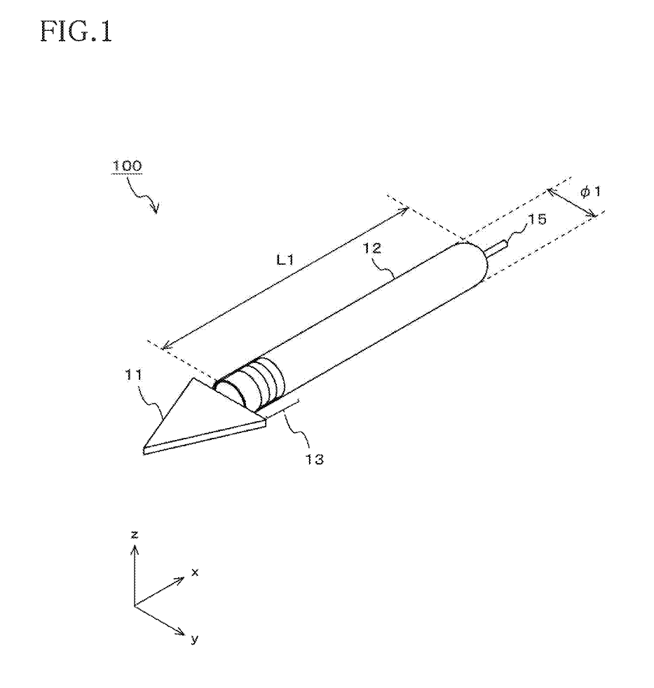

The following will describe a surgical fluid-absorbing instrument and a method of producing the same according to this invention with reference to the drawings. The surgical sponge 100 shown in FIG. 1 constitutes an example of the surgical fluid-absorbing instrument and absorbs blood occurring in an ophthalmologic surgery or the like using a microscope and/or surgical fluid (waste fluid) used for washing the relevant wound.

The surgical sponge 100 has a tip sponge part 11, a handle part 12 and a retaining sponge part 13. The tip sponge part 11 constitutes an example of the first fluid-absorbing part and absorbs the blood occurring in the surgery and/or fluid (moisture) such as the surgical waste fluid used for washing the relevant wound. The tip sponge part 11 has a previous state before use, namely, a state in which it is dried without absorbing any fluid and solidified. It has a shape which is identical to, for example, an isosceles triangle shown in FIG. 1. Of course, the solidified shape is not limited to the isosceles triangle; it may be a regular triangle.

To the tip sponge part 11, the handle part 12 is attached. The handle part 12 constitutes an example of the retaining member and has a long and narrow tubular shape (straw shape). It is made by making the conventional toothpick-like handle thicker and hollow. In this embodiment, it is configured that the handle part 12 does not only function as a water-absorbing tank but also moves the fluid absorbed from the tip rearward within the handle part 12. In the drawing, L1 indicates a length of the handle part 12 and φ1 indicates an inside diameter thereof. A terminal of the handle part 12 is squeezed and is provided with a drain port 15 at the terminal thereof. The drain port 15 is provided for draining the fluid retained in the handle part 12 to the outside utilizing the drain port 15.

Dimensions of the handle part 12 vary according to purposes of surgery or the like. When a surgical field is a surface of the relevant wound, the length L1 thereof is, for example, about 5 through 10 cm and the inside diameter φ1 thereof is about 5 through 10 mm. When a surgical field is a profound relevant wound, the length L1 thereof is, for example, about 10 through 30 cm and the inside diameter φ1 thereof is about 10 through 20 mm. A cross-sectional shape of the handle part 12 is not limited to a circular shape; it may be a polygonal shape such as a cross-sectional square. The handle part 12 also may be an oblong shape (an oval type) in planar view.

The retaining sponge part 13 is loaded into the handle part 12 so that it is connected to the tip sponge part 11. The retaining sponge part 13 constitutes the second fluid-absorbing part and absorbs the fluid. The retaining sponge part 13 is provided for moving the fluid or the like absorbed by the tip sponge part 11 to another place.

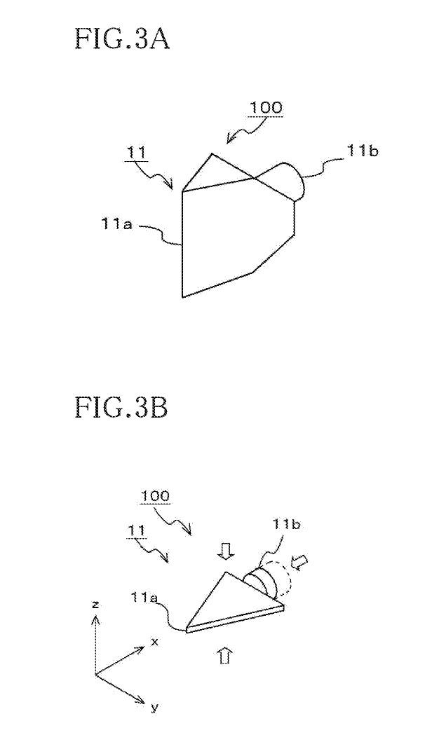

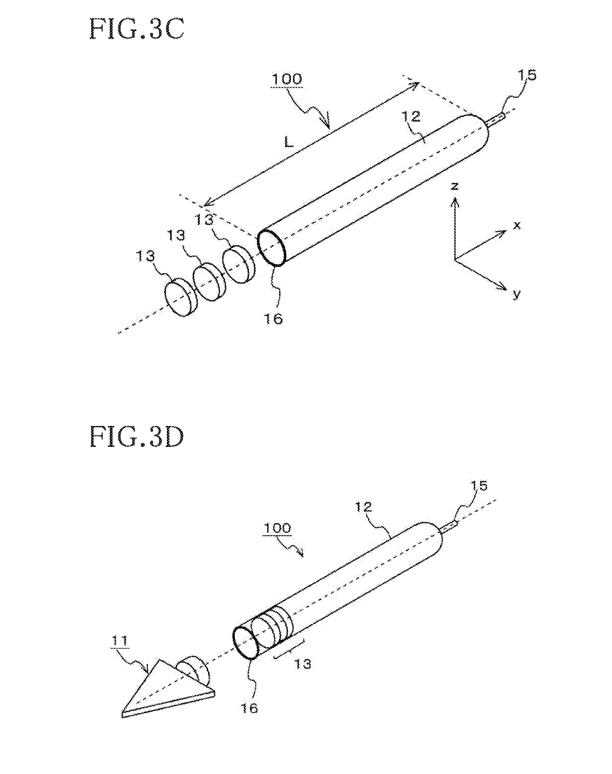

The retaining sponge part 13 has a previous state before use, namely, a state in which it is dried without absorbing any fluid and solidified similar to the tip sponge part 11. It has, for example, a column shape (a coin shape) as shown in FIGS. 3C and 3D. Of course, the solidified shape thereof is not limited to the coin shape: It may be a shape that is almost identical to an inside diameter of the handle part 12. An x direction in a system of x, y and z coordinates shown in FIGS. 3B and 3C indicates a tube axis direction of the handle part 12, a y direction therein indicates a radical direction thereof and a z direction therein indicates a direction that is perpendicular to the x and y directions.

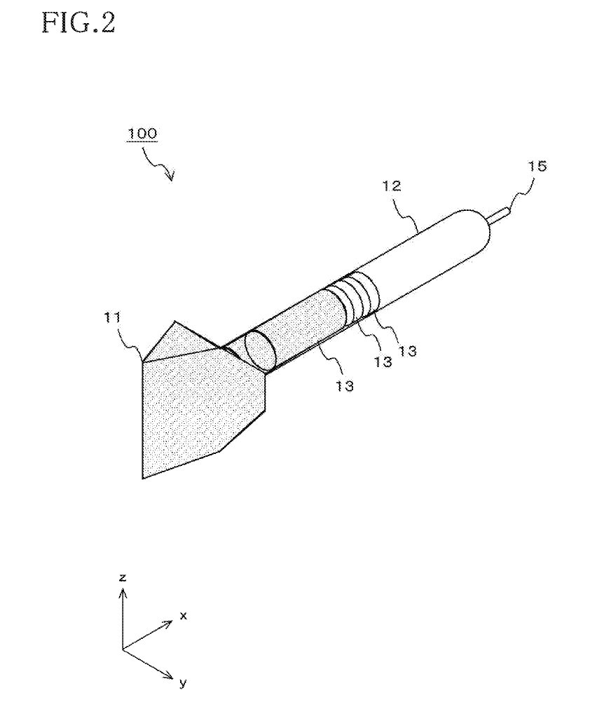

The surgical sponge 100 shown in FIG. 2 has a state where it absorbs the fluid and is swollen. In the drawing, a portion absorbing the blood and/or fluid such as surgical waste fluid, not shown by a mark, is indicated like pearskin. In FIG. 2, the tip sponge part 11 and the retaining sponge part 13 are composed of anisotropic water-absorbing sponges. Most of the water-absorbing sponges have a nature in which when the dried one absorbs fluid (water), it swells to a fixed direction (isotropic direction).

For the water-absorbing sponge, PVA sponge material molded by chemically reacting hydrous polymeric material such as polyvinyl alcohol (PVA) is used. The PVA sponge material has very excellent water absorbing nature and its micropores cause capillary phenomenon so that it has water holding property and has both of flexibility and suitable elasticity. The PVA sponge does not wound a target surface. Of course, the water-absorbing sponge material is not limited to PVA sponge in which polyvinyl alcohol is raw material: It may be PVA sponge in which polyvinylacetal is raw material. The latter is a water-absorbing sponge having porous structure and smooth surface and can absorb the fluid 25 times weight of a manufactured article. PVA-based sponge material is compressed and hardened when it is dried.

The water-absorbing sponge material is not limited to PVA-based one: Urethane-based sponge material or cellulose-based sponge material may be used. The urethane is light-weight and is rich in cushion elasticity. The narrower a pore is, it absorbs water. Since hydrophilic urethane has a hydrophilic group, it quickly absorbs water to be swollen. Polyurethane is porous to have continuous porous. Polyurethane is rich in hydrophilicity more than general urethane and is excellent in water absorbing and preserving capacity.

Since cellulose has a hydrophilic group, it is easy to harmonize the water. Capillary phenomenon by its micropores exhibits self-water-absorbing quality and quickly absorbs the water without compulsion. The cellulose has water-absorbing quality that is 20 through 30 times its own weight to preserve the water suitably. It is rare to drop the absorbed water from the cellulose. The cellulose has a soft surface with any suitable elasticity at a wet time thereof and is compressed and hardened to about 60% at a dried time thereof.

By utilizing the nature in a swelling direction, the retaining sponge part 13 is set so as to be swollen rearward within the handle part 12. The anisotropic retaining sponge part 13 is easy to be made by cutting normal (isotropic) water-absorbing sponge material to be made long column shaped, loading the cut water-absorbing sponge material in a visible tubular material having a termination at an end thereof, and compressing it from the other end, drying and solidifying it. Thus, the water-absorbing sponge that has been compressed from one direction, dried and solidified is swollen to the one direction when absorbing the fluid (water) or being not dried nor solidified to return its long column shaped water-absorbing sponge material. By utilizing this nature, it is possible to swell the water absorbed retaining sponge part 13 rearward (to one direction) within the handle part 12 and smoothly move the fluid rearward within the handle part 12.

The following will describe the method of producing the surgical sponge 100 with reference to FIGS. 3A through 3D. In this embodiment, it is an assumption that the surgical sponge 100 shown in FIG. 1 is produced. In this production method, the tip sponge part 11 is composed of a first fluid-absorbing member in which one of the water-absorbing sponge material is compressed on one direction, and the retaining sponge part 13 is composed of a second fluid-absorbing member in which the other of the water-absorbing sponge material is compressed on a direction that is perpendicular to the compressed direction of the tip sponge part 11. A case in which, in this case, the first and second fluid-absorbing members are connected to each other with a direction (hereinafter, simply referred to as “compression direction”) of the compression of the first fluid-absorbing member being perpendicular to a direction (hereinafter, simply referred to as “compression direction”) of the compression of the second fluid-absorbing member is exemplified. The compression direction of the retaining sponge part 13 is a longitudinal direction of the handle part 12.

First, the tip sponge part 11 shown in FIG. 3A is prepared. For example, PVA water-absorbing sponge material is processed to have a desired dimension. In this embodiment, the tip sponge part 11 is formed. The tip sponge part 11 has a spatial structure such that a tip portion 11a like a triangular prism with a sloped surface, which shows a deformed hexagon (hoe shape) viewed from a side thereof, in a state where it does not absorb water, and an isosceles-triangle viewed from an upper surface thereof, is combined to a column attaching portion 11b at a rear surface side of the tip portion 11a. The tip sponge part 11 having the spatial structure is formed using a punch-out processing device or a molding processing device. The attaching portion 11b is processed to be made column shaped so that it has an outer diameter which is almost identical to an inside diameter of the handle part 12.

Next, the tip sponge part 11 having the spatial structure shown in FIG. 3A is compressed, dried and solidified to form the anisotropic tip sponge part 11 (first fluid-absorbing member) shown in FIG. 3B. In order to solidify the water-absorbing sponge material, water-soluble adhesive which is harmless for eyeball is used. The tip portion 11a is compressed, dried and solidified on the z direction. The attaching portion 11b is compressed, dried and solidified on the x direction that is perpendicular to the z direction. This is because the water-absorbing sponge of the attaching portion 11b is swollen to the x direction. The anisotropic tip sponge part 11 can be easily formed by using a pressing machine, having compressing function, in which a nipping direction and a pushing direction are different from each other.

It is to be noted that the compressing, drying and solidifying steps of the attaching portion 11b on the x direction may be omitted and the column shape thereof in the state where it does not absorb water may be remained as it is. Further, when it is difficult to combine the tip portion 11a to the attaching portion 11b as described above, a narrow tube may be provided to skewer (piece) both of the triangular tip portion 11a and the attaching portion 11b and use the capillary phenomenon of the tube.

Next, the retaining sponge part 13 is attached to the handle part 12 shown in FIG. 3C. The handle part 12 which has an open end 16 and the other squeezed end and is provided with the drain port 15 at this squeezed portion thereof is prepared. For the visible material of the handle part 12, a flexible tube having at least any one quality of reversibility and flexibility may be used. It is more preferable that for the handle part 12, a resin pipe such as straw material may be used and a transparent material through which an internal portion thereof is observable (visible) may be used.

On the other hand, water-absorbing material is processed to have a desired dimension to form the retaining sponge part 13. In this embodiment, the water-absorbing material in a state where it does not absorb water is processed to be a long and narrow column shape. For example, for swelling the retaining sponge part 13 when it absorbs water to be moved rearward, the water-absorbing material is cut so that it has an outer diameter that is just smaller than the inside diameter of the handle part 12. This dimensioning is because it is made easy to insert the retaining sponge part 13 into the handle part 12 (outer casing) and to swell and wring the same. A length of the retaining sponge part 13 is, for example, about 20 mm in a state where it does not absorb any water.

In this embodiment, a case where three retaining sponge parts 13 are loaded in the handle part 12 having a length L1 of 6 cm is exemplified. By compressing, drying and solidifying the column shaped water absorbing material having a height of about 20 mm and an outer diameter of about 6 mm to a thickness of about 4 mm, the anisotropic retaining sponge parts 13 (second fluid-absorbing member) shown in FIG. 3C is formed. More than one retaining sponge part 13 thus configured, for example, three ones are prepared and they are loaded into the handle part 12 from a side of the open end 16. A thickness of the retaining sponge part 13 may be an extent preventing the retaining sponge part from being rotated in the visible material of the handle part 12.

Of course, the outer diameter of the retaining sponge part 13 is not limited to 6 mm, its thickness is allowed to be an extent such that it is adhered to an interior wall of the handle part 12 when swollen and it does not move. The length of the retaining sponge part 13 is not limited to a tripartite length of the handle part 12: The retaining sponge part 13 may be one water-absorbing sponge material having a length that is identical to the length L1 of the handle part 12 of 6 cm (L1=6 cm). In its compressing processing thereof in this case, it is possible to form the anisotropic large capacity retaining sponge part 13 by loading water-absorbing sponge material having the same as the inside diameter of the handle part 12 into the visible material having one terminal at its end and compressing, drying and solidifying it from the other side.

For example, three retaining sponge parts 13 in their dried and compressed state are loaded into the visible material of the handle part 12 with their anisotropy being aligned. The alignment of their anisotropy allows the retaining sponge parts 13 each absorbing fluid to be successively swollen to one direction, thereby enabling the fluid to be successively moved to one direction. In contrast, in a case of the large capacity retaining sponge part 13, a work of aligning their anisotropy may be omitted.

Next, the tip sponge part 11 prepared regarding FIG. 3B is attached to the handle part 12 shown in FIG. 3D. In this embodiment, the attaching portion 11b of the tip sponge part 11 is attached to the open end 16 of the handle part 12 so that the attaching portion 11b can contact the retaining sponge part 13. This enables to be completed the surgical sponge 100 in which the retaining sponge part 13 having the same function as that of the tip sponge part 11 contacts the tip sponge part 11 within the straw-like hollowed handle part 12.

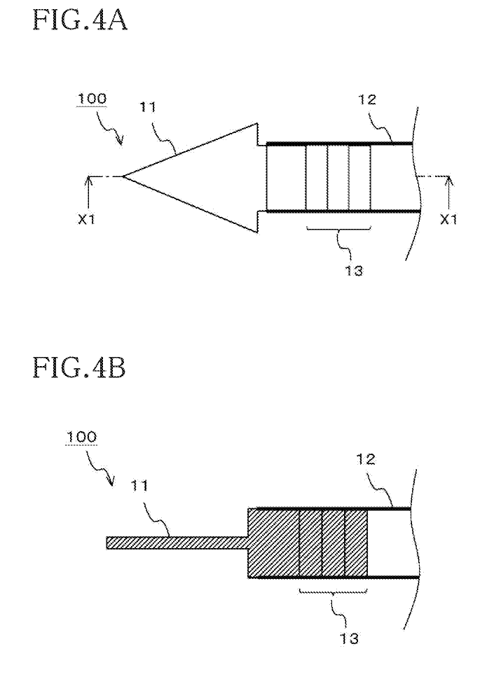

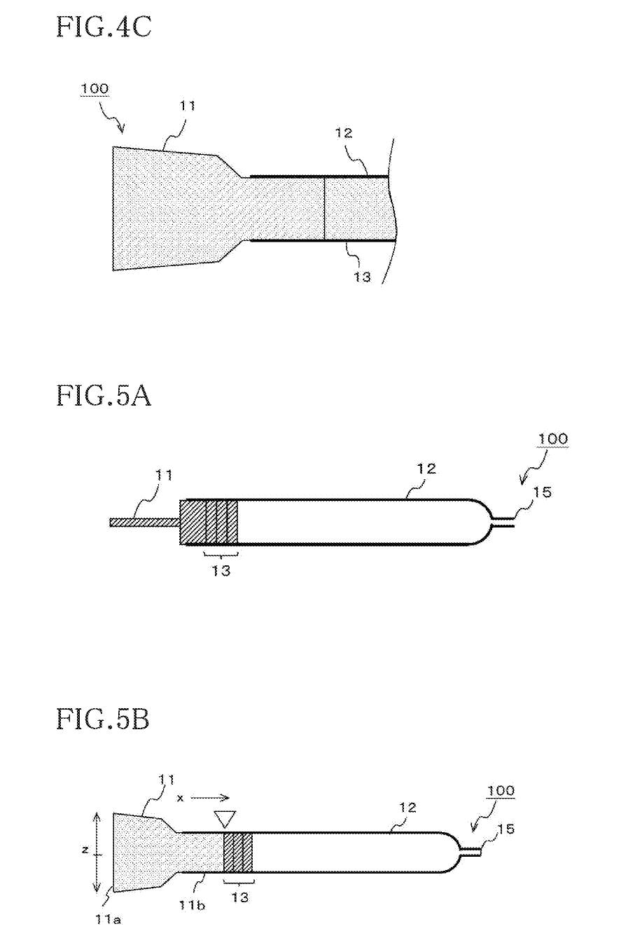

The following will describe a function example of the surgical sponge 100 with reference to FIGS. 4A through 6B. FIGS. 4A and 4B are sectional views of the surgical sponge 100 viewed from an upper surface and a side surface in a state where it does not absorb any water and FIG. 4C is a sectional view thereof viewed from the side surface in a state where it absorbs water.

The surgical sponge 100 has two modes of a state in which the tip sponge part 11 and the retaining sponge part 13 are compressed on one direction and dried as shown in FIGS. 4A and 4B and a state in which the tip sponge part 11 and the retaining sponge part 13 are extended on one direction and swollen as shown in FIG. 4C.

Thus, the states of the surgical sponge 100 before and after the use thereof can be easily distinguished from the state before the tip sponge part 11 and the retaining sponge part 13, which have been dried and compressed state as shown in FIGS. 4A and 4B, absorb fluid and the retained state after the tip sponge part 11 and the retaining sponge part 13, which are water-retaining and swollen states as shown in FIG. 4C, absorb the fluid.

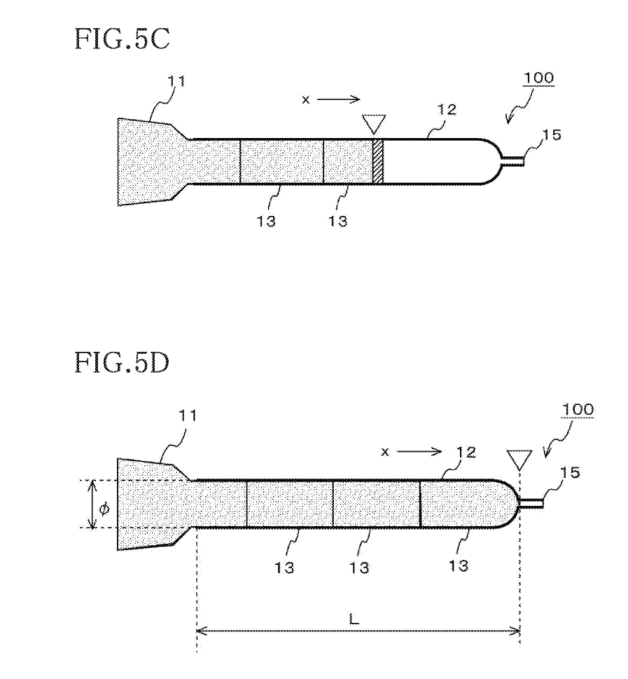

The following will describe a checking example of an amount of absorbed water by the surgical sponge 100 with reference to FIGS. 5A through 5D. In this embodiment, the handle part 12 is composed of visible material through which an internal portion thereof is transparently seen. For example, the visible material is set to be straw material and to be made transparent or semitransparent. FIG. 5A shows a state of the surgical sponge 100 before the use thereof. This state has been shown in FIGS. 4A and 4B.

FIG. 5B shows a state where the fluid is swollen in the tip portion 11a and the attaching portion 11b after a start of use in the surgical sponge 100. Under this state, the tip portion 11a is swollen to the z direction to form a triangular prism with a sloped surface (see FIG. 3A). The attaching portion 11b is swollen to the x direction to push the first retaining sponge part 13. In the drawing, a triangle mark defined by outlines is an indicator for checking volume and indicates a position near a boundary between the attaching portion 11b and the first retaining sponge part 13 by which a fluid-moving state can be checked. This is a position indicating almost whole volume of the handle part 12. This indicates allowed volume in which the handle part 12 can contain the fluid.

FIG. 5C shows a state where the first retaining sponge part 13 and a half of the second retaining sponge part 13 are swollen by the fluid to be moved to the x direction after the further use of the surgical sponge 100. In the drawing, the triangle mark defined by outlines indicates a position near a boundary of the first retaining sponge part 13 by which a fluid moving state can be checked. This is a position indicating a half volume in which the handle part 12 contains the fluid. This position indicates remained allowable half volume.

FIG. 5D shows a state where all of the three retaining sponge parts 13 are swollen by the fluid to be moved to the x direction after the still further use of the surgical sponge 100 and the third retaining sponge part 13 reaches the terminal portion of the handle part 12. Thus, when the fluid absorbed from the tip sponge part 11 is moved to the retaining sponge part 13 in the handle part 12, the following retaining sponge parts 13 are successively swollen rearward. This allows viewing how much the fluid is absorbed and how much the fluid can be still absorbed.

Here, when the inside diameter φ1 of the visible material (a straw or the like) of the handle part 12 shown in FIG. 5D is set to be 6 mm and the length L1 thereof is set to be 6 cm which is identical to the length of the present toothpick like handle, the amount of absorbed water is about 1.7 ml. Since according to the conventional system, the water absorption rate is about 0.2 through 0.3 ml, this is the amount of absorbed water 5 through 8 times that of the surgical sponge according to the conventional system. This enables the present problem of shortage in the amount of absorbed water to be solved.

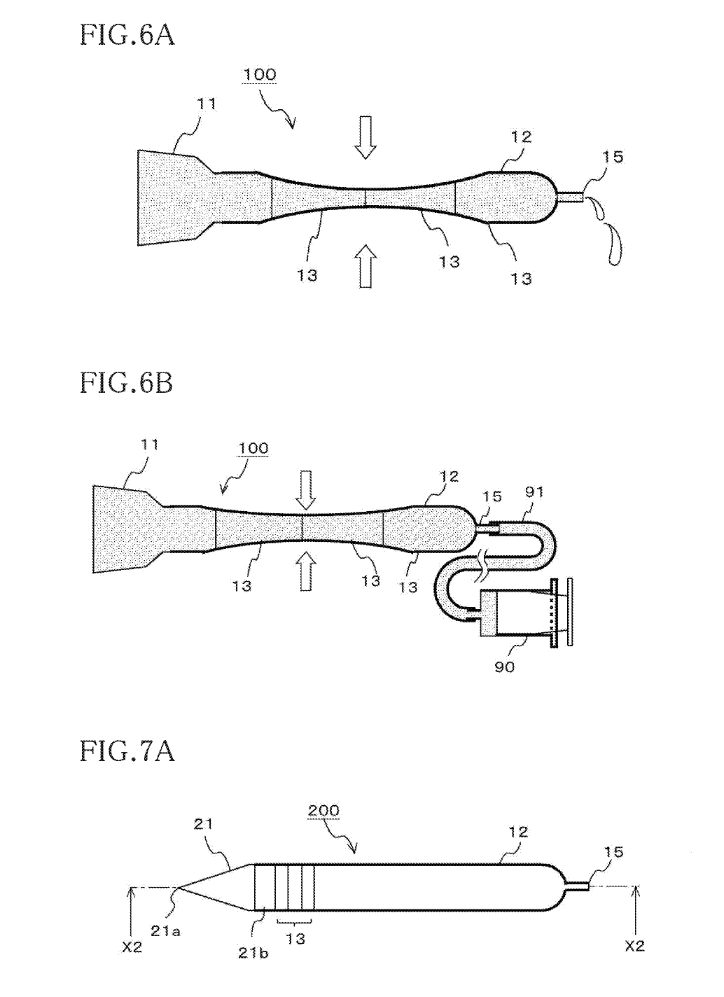

Here, the following will describe a drain example of the fluid from the surgical sponge 100 with reference to FIGS. 6A and 6B. In this embodiment, the visible material of the handle part 12 is configured to be a flexible tube having at least any one quality of reversibility and flexibility. This enables the visible material to be squeezed by applying any force from the outside of the handle part 12 as shown in FIG. 6A.

The use of such visible material allows the fluid retained in the handle part (water storage tank) to be easily drained from the drain port 15 provided at a rear end of the handle part 12. Therefore, the handle part 12 and the retaining sponge part(s) 13 can be repeatedly used so that the fluid is absorbed by the retaining sponge part(s) 13 by utilizing capillary phenomenon and drained to the outside. Any extra effort and a check like the conventional system such that by pushing it against other cloth or the like, an amount of fluid in the water-absorbing sponge is adjusted, and the check is performed on whether or not any fibers and/or foreign matters are adhered to the water-absorbing sponge become unnecessary.

A long hose (tube) 91 shown in FIG. 6B is connected to the above-mentioned drain port 15 according to a purpose of the surgery. When the hose 91 or the like is connected to the drain port 15, such a usage to control the draining path can be adopted. Using the hose 91 allows the fluid to be drained and led to a lower position by applying principal of siphon. Accordingly, after the retaining sponge parts 13 absorb water to lengthen, it is possible to absorb the water as the normal sponge by utilizing the capillary phenomenon and perform a continuous water draining job together with the pump function. Further, it is possible to lead the fluid retained in the handle part 12 to a distant place so that the fluid retained in the handle part 12 can be drained to a predetermined place away from the relevant wound.

In this embodiment, a syringe 90 may be used so as to be connected to a terminal end of the hose 91 or the like. The syringe 90 constitutes an example of a drainage device and forcibly evacuates the fluid retained in the handle part 12. Such a configuration allows the fluid retained in the handle part 12 to be evacuated to a place away from the relevant wound.

The drainage device is not limited to the syringe 90: A pipette, a suction tube, an electric pump and the like may be used. Of course, when the hose 91 is omitted, the syringe 90 is directly connected to the drain port 15 of the handle part 12 and a nursing assistant or the like performs an evacuation job and the like, it is possible to contain the fluid absorbed by the surgical sponge 100 in the syringe 90 without going through the hose 91. The fluid contained in the syringe 90 can be transferred to an evacuated inspection tube.

Combination of the above-mentioned drainage device enables an amount of drained fluid to be increased so that it is possible to provide the surgical sponge 100 which is convenient and is very versatile, thereby making compatible for both of corneal surgery in which the field of surgery is the surface of relevant wound and coronary artery surgery or the like in which the field of surgery is the profound wound. It is needless to say that a change of the surgical sponge 100 can be avoided in the surgery.

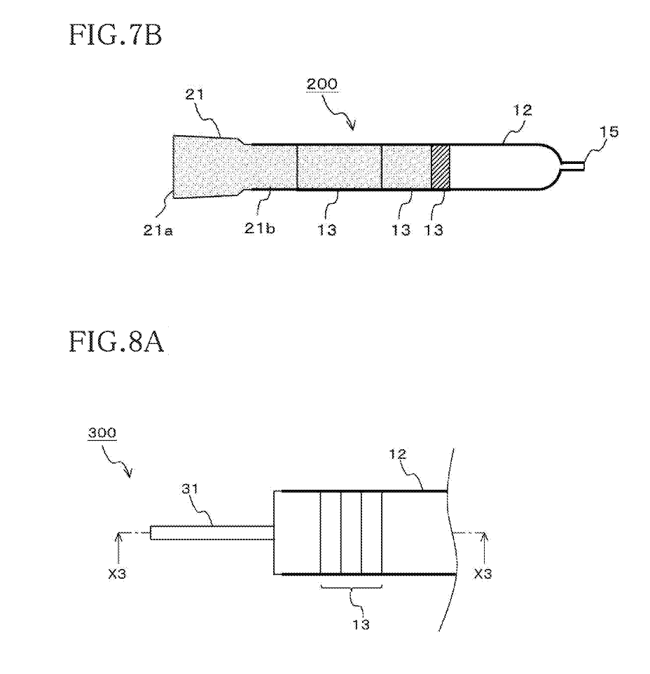

Next, the following will describe a configuration example of a surgical sponge 200 as a variation example with reference to FIGS. 7A and 7B. The surgical sponge 200 shown in FIG. 7A has a narrower tip sponge part 21 than that of the surgical sponge 100 shown in FIG. 1. A tip 21a is narrow and an attaching portion 21b has the same thickness as that of the attaching portion 11b of the surgical sponge 100. The surgical sponge 200 is preferably available for washing a restricted relevant wound.

As to the tip 21a, by using any material having small swelling rate for water-absorbing sponge, it is possible to make a smaller tip sponge part 21, not with a conventional configuration such that the tip is swollen. Of course, solidity of the water-absorbing sponge may alter without changing a dimension thereof. Description of other configurations shown in FIG. 7B will be omitted because they have the same configuration and function as those of the handle part 12, the retaining sponge part 13 and the like of the surgical sponge 100.

In the ophthalmologic surgery using a microscope, the surgical sponge 200 can restrict its expanded volume to have the expanded volume smaller than that of the surgical sponge 100 even when it absorbs the fluid and swollen so that it is possible to prevent visibility of the relevant wound from being deteriorated. This enables the operator's visibility in the surgery to be improved.

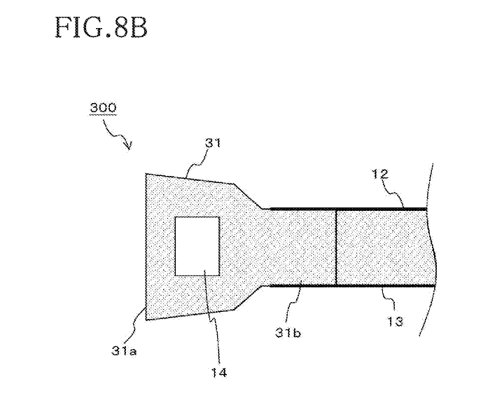

Further, the following will describe a configuration example of a surgical sponge 300 as another variation example with reference to FIGS. 8A and 8B. The surgical sponge 300 shown in FIG. 8A has a tip sponge part 31 in a dried and compressed state, which has the same shape as that of the tip sponge part 11 of the surgical sponge 100 shown in FIG. 1 in a dried and compressed state. As shown in FIG. 8B, in a state where the water-absorbing sponge constituting the tip sponge part 31 is expanded to one direction and swollen, a window 14 passing through the water-absorbing sponge is provided.

By providing with the window 14, even if the volume of the tip sponge part 31 is less than that of tip sponge part 31 before the window 14 is opened, an amount of absorbed water has never dropped because the retaining sponge part 13 is provided in the handle part 12. Of course, since the relevant wound at an opposite side of the window 14 is seen from the window 14, it is possible to perform the washing (water retaining) operations while looking at a state of the relevant wound.

For example, the surgical fluid is absorbed by gently sliding the surgical sponge 100 or the like along a surface of the eyeball. Thereby, a method of enabling the fluid absorbed by the tip sponge part 11 to be moved to the retaining sponge part 13 within the handle part 12 may be adopted. According to this method, any hose or tube for absorbing the absorbed fluid and moving it to another place by the pump or the like is unnecessary so that its operation has never deteriorated.

By adopting this method, water absorbency of the tip sponge part 11 enables water-absorbing operation to be spread until the water-absorbing rate of the retaining sponge part 13 in the handle part 12 reaches the limit thereof. Accordingly, it is possible to design the tip sponge part 11 to be smaller (narrower) than that of the conventional system, in order to maintain visibility of the operator. It is also possible to avoid a situation in which many surgical sponges 400 are necessary in the surgery like the conventional system.

Thus, according to the surgical sponge 100 as the first embodiment, it is so configured that the retaining sponge part(s) 13 provided at a position which is continuous from the tip sponge part 11 is loaded into the handle part 12 to absorb the fluid.

Under this configuration, since the fluid absorbed from the tip sponge part 11 can be stored in the handle part 12, it is possible to absorb the fluid some time the conventional surgical sponge 100. This enables changing times of the surgical sponge 100 in the surgery to be considerably decreased.

According to the above-mentioned surgical sponge 100, since the water-retaining state of the retaining sponge part(s) 13 in the handle part 12 is seen from the outside thereof, it is possible to perform the washing (water retaining) operation while checking water-retaining state of the handle part 12.

Moreover, according to the surgical sponge 100, the fluid absorbed from the tip sponge part 11 and stored in the retaining sponge part(s) 13 is moved from a side of the tip sponge part 11 to the drain port 15 proved at a rear end. Further, by squeezing the flexible tube (visible material) from the outside, the fluid contained in the retaining sponge part(s) 13 therein can be disposed to the outside. Therefore, the retaining sponge part(s) 13 in the handle part 12 can be used over and over.

According to the method of producing the surgical sponge 100, it is also so configured that after the anisotropic retaining sponge part(s) 13 is (are) loaded in the tubular handle part 12, the anisotropic tip sponge part 11 is loaded in the handle part 12 from a side of the open end 16 so as to contact the retaining sponge part(s) 13.

By this configuration, it is possible to produce the surgical sponge 100 which can store the fluid absorbed from the tip sponge part 11 in the handle part 12 with excellent reproducibility. This enables the surgical sponge 100 which can absorb a large amount of surgical fluid or the like to be presented.

Further, when such a configuration that the handle part 12 has both open terminal ends, not squeezed terminal end, an order of lading and attaching the tip sponge part 11 and the retaining sponge part(s) 13 may alter. For example, such a method of loading the retaining sponge part(s) 13 in the handle part 12 so that it (they) contacts the tip sponge part 11, after the tip sponge part 11 is previously attached to the tubular handle part 12, may be adapted. It is also possible to use the handle part 12 according to this invention in place of the sickle-shaped portion of the conventional apparatus which absorbs water (see the above-mentioned US patent).

Although the case where the tip sponge part 11 and the retaining sponge part 13 are configured to be two water-absorbing materials has been described in the above-mentioned embodiment, this invention is not limited thereto: It is composed of a water-absorbing sponge in which the tip sponge part 11 and the retaining sponge part 13 are integrated. As a method of producing it, one end of one of the water-absorbing sponge is compressed from a desired direction to be a compressed and solidified shape so that the tip sponge part 11 is formed on the one end thereof. Next, the water-absorbing sponge is compressed in a direction that is perpendicular to the compressed direction of the tip sponge part 11 to be a compressed and solidified shape so that the retaining sponge part 13 is formed on the other end thereof. When the surgical sponge 100 is thus configured, its assembly becomes made easy.

### Second Embodiment

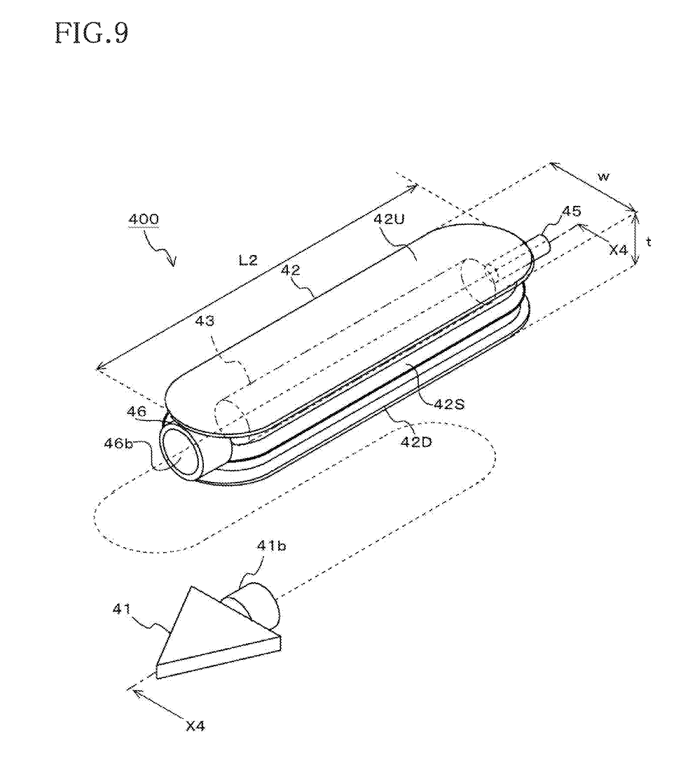

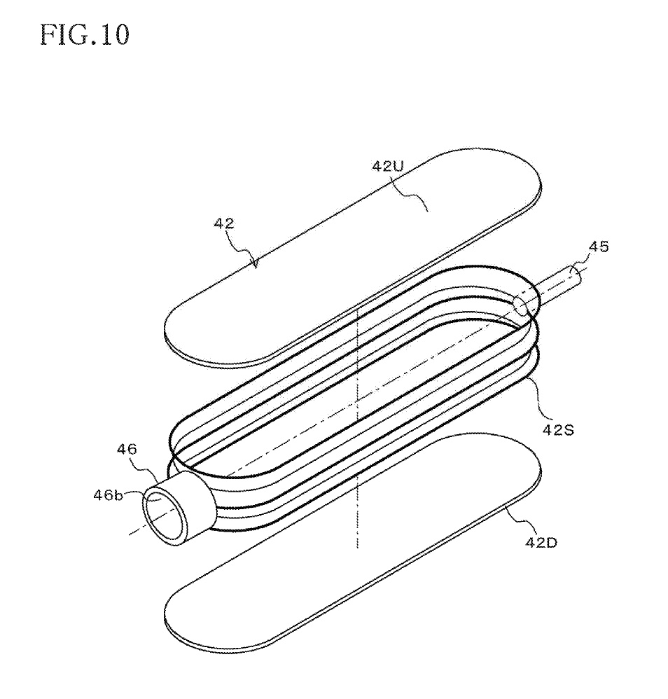

The following will describe a handy surgical sponge 400 of bellows operating type as a second embodiment with reference to FIGS. 9 through 11C. The surgical sponge 400 shown in FIG. 9 constitutes an example of the surgical fluid-absorbing instrument and absorbs blood occurring in an ophthalmologic surgery or the like using a microscope and/or surgical fluid (waste fluid) used for washing the relevant wound. The surgical sponge 400 has a handle part 42, which is different from the handle part 12 described in the first embodiment, having a bellows structure which can expand and contract to, at least, a direction that is perpendicular to a longitudinal direction of the visible material.

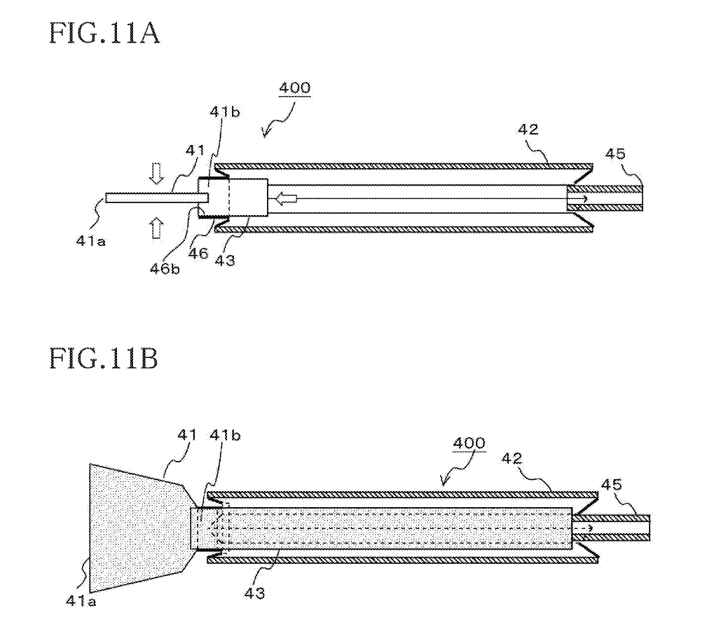

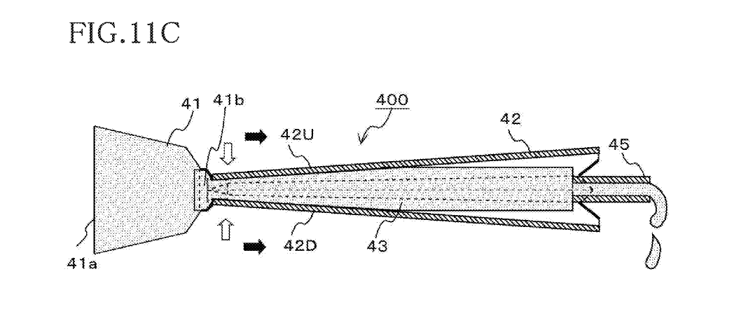

The surgical sponge 400 has a tip sponge part 41, the hollow handle part 42 which attaches the tip sponge part 41 and a retaining sponge part 43 to be stored in the handle part 42 (see FIG. 11B), an drain port 45 and an open end 46, which are provided in the handle part 42. In the drawing, L2 indicates a length of the handle part 42, “w” indicates a width thereof, and “t” indicates a thickness thereof. A dimension of the handle part 42 is different from each other according to the use of surgery. For example, the length L2 thereof is about 5 through 8 cm, the width w thereof is 1.0 through 3.0 cm and the thickness t thereof is about 0.5 through 3.0 cm. Similar to the first embodiment, in this embodiment, L2 is 6 cm, and “w” is about 2 cm and “t” is about 1 cm. In this case, an amount of absorbed water of the surgical sponge 400 is about 12 ml. This enables its amount of absorbed water to be increased more than that of the surgical sponge 100.

The tip sponge part 41 can use the first fluid-absorbing material which is identical to the tip sponge part 11 described in the first embodiment. Since its details have been described on the tip sponge part 11, they will be omitted. A rear end of an attaching portion 41b of the tip sponge part 41 is attached into an open hole 46b of the open end 46 of the handle part 42 with it being adhered by a water-soluble adhesive to a forward end of the retaining sponge part 43 shown in FIG. 11A.

The handle part 42 shown in FIG. 10 is of bellow operating type and constitutes an example of retaining member. In this embodiment, the handle part 42 is composed of visible material through which an internal portion thereof is transparently seen. The handle part 42 is provided with a bellows portion 42S having a bellows structure which is parallel with the longitudinal direction of the handle part 42. The bellows portion 42S shows a tubular (track field like) shape with a square and round rectangular in planar view. The bellows portion 42S preferably has three to five bellows to deal with the surgical sponge 400 by a single hand of a user when the surgical sponge 400 absorbs or drains the fluid. The invention, however, is not limited thereto.

Pushing plates 42U, 42D are attached to upper and lower open ends of the bellows portion 42S. Each pushing plate has the same dimension and shape as those of each of the upper and lower open ends. In this embodiment, the bellows portion 42S of the handle part 42 has any one quality of reversibility and flexibility. Accordingly, the bellows portion 42S is squeezed when the pushing plates 42U, 42D are nipped by a hand and any force is applied thereto.

A column like open end 46 is provided at one arc portion (a corner of the track field) of the bellows portion 42S. The drain port 45 is provided at the other arc portion of the bellows portion 42S.

As shown in FIG. 11A, the retaining sponge part 43 is loaded into the handle part 42 so that it is connected to the tip sponge part 41. The retaining sponge part 43 constitutes the second fluid-absorbing part and absorbs the fluid. Many pores each having an inside diameter of about some mm are cut at an end portion of the retaining sponge part 43 which is connected to the tip sponge part 41 in order to increase water absorption and permeability.

The retaining sponge part 43 has a nature such that it is a compressed state before it absorbs the fluid but it expands (swells) to a fixed direction when it absorbs the fluid. The retaining sponge part 43 before it absorbs the fluid has a compressed (dried) and solidified state, which is similar to the tip sponge part 41, and its shape is, for example, a column-like shape (with its long side surface). Of course, the shape, when solidified, is not limited to the column shape: A rectangular parallelepiped shape, a square and round rectangular shape and the like are available if they could be contained in the handle part 42.

The retaining sponge part 43 shown in FIG. 11B has a state where it absorbs the fluid and is swollen. In the drawing, a portion absorbing the blood and/or fluid such as surgical fluid, not shown by a mark, is indicated like pearskin. The tip sponge part 41 and the retaining sponge part 43 are composed of anisotropic water-absorbing sponges. The retaining sponge part 43 swells toward only a longitudinal direction of the handle part 42 along an internal surface of the handle part 42 when a compressed and dried retaining sponge part absorbs the fluid (water). Thus, the handy surgical sponge 400 of bellows operating type is configured.

The following will describe a method of producing the surgical sponge 400 with reference to FIGS. 9 through 11A. First, the tip sponge part 41 shown in FIG. 9 is prepared. Since the method of producing the tip sponge part 41 has been described in the first embodiment, it will be omitted.

Next, the handle part 42 shown in FIG. 10 is prepared. For the bellows portion 42S, a die is prepared to mold the bellows having the open end 46 for entering the fluid and the drain port 45 for draining the fluid to be made square and round rectangular. A terminal endless tube made of silicon resin is set into the die under aseptic condition and by blowing air from the other terminal, it is molded so as to be made square and round rectangular. For the bellows portion 42S, a material such as plastic resin, which has at least any one quality of reversibility and flexibility, may be used. It is more preferable that a transparent material through which an internal portion thereof is transparently observable (visible).

The track-field-like pushing plates 42U, 42D are connected to the square and round rectangular (track-field-like) open ends of the bellows portion 42S as an upper lid and a lower lid. The pushing plates 42U, 42D may use plates such as rigid vinyl, plastics and the like, which are molded to be made track-field-like shaped. It is to be noted that if the bellows portion 42S and the pushing plates 42U, 42D are made from the same material, the bellows portion 42S and the pushing plates 42U, 42D may be molded by integral molding.

On the other hand, the water-absorbing material is cut so that the retaining sponge part 43 shown in FIG. 11A is just smaller than the inner side of the handle part 42. The handle part 42 of this embodiment has a length of 6 cm, a width of 2 cm and a thickness of 1 cm. This dimensioning is because it is made easy to insert the retaining sponge part 43 into the handle part 12, to swell the retaining sponge part 43 when it absorbs water and to drain the water.

The water-absorbing material is then compressed, dried and solidified to be about 6 mm and a rectangular anisotropic retaining sponge part 43 as shown in FIG. 11A is formed. Many pores having a diameter of about some mm are cut at an end portion of the retaining sponge part 43 which is connected to the tip sponge part 41. As a result thereof, the retaining sponge part 43 has a partially porous structure. The tip sponge part 41 and the retaining sponge part 43 are connected to each other by water-soluble adhesive. The tip sponge part 41 and the retaining sponge part 43 thus connected are loaded into the handle part 42 from a side of the open end 46.

As to a loading direction, for example, it is loaded so that a plane surface of the tip sponge part 41 when it is dried and solidified and a plane surface of the handle part 42 becomes the same plane surface. When loading them like this, the plane surface of the swollen tip sponge part 41 and the plane surface of the handle part 42 are perpendicular to each other, so that the handle part 42 is nipped with it being pinched by the thumb and the index finger, it is possible to improve sweep operability and workability on the field of surgery. The attaching portion 41b of the tip sponge part 41 is inserted into the open hole 46b of the open end 46.

Thus, the surgical sponge 400 in which the tip sponge part 41 and the retaining sponge part 43 are connected to each other in the handle part 42 is completed.

The following will describe a functional example of the surgical sponge 400 with reference to FIGS. 11A through 11C.

(1) When the absorption of the fluid starts, the tip sponge part 41 shown in FIG. 11A swells by osmotic pressure.
(2) The fluid absorbed from the tip sponge part 41 is permeated to a side of the attaching portion 41b.
(3) The attaching portion 41b is swollen by the permeated fluid and when the attaching portion 41b pushes the retaining sponge part 43, the fluid is further permeated into the retaining sponge part 43 to swell the retaining sponge part 43.
(4) A swollen state of the retaining sponge part 43 is easily seen by visual inspection from the outside of the handle part 42 so that it is possible to check a permeated state of the fluid. Therefore, it is possible to visually confirm how much the fluid is absorbed or how much it could be still absorbed.
(5) As shown in FIG. 11B, all of the tip sponge part 41 and the retaining sponge part 43 is filled with the fluid.
(6) A user nips the pushing plates 42U, 42D of a side of the attaching portion 41b as shown in FIG. 11C with his hand to apply any force to directions of arrows defined by outlines.
(7) By applying the force to the pushing plates 42U, 42D, the bellows portion 42S is squeezed so that the fluid within the retaining sponge part 43 is flown out toward a side of the drain pot 45. Further, a part of the fluid is flown backward to a side of the tip sponge part 41.
(8) When the user moves a position to which the force should be applied to a side of the drain port 45 (to a direction shown by black arrows in the drawing) little by little, the absorbed fluid is drained from the drain port 45.

Thus, the surgical sponge 400 takes two modes of a state in which the tip sponge part 41 and the retaining sponge part 43, as shown in FIG. 11A, are compressed and dried and a state in which the tip sponge part 41 and the retaining sponge part 43, as shown in FIG. 11B, absorb the fluid and swollen. Accordingly, these two modes enable the states of the surgical sponge 400 before and after the use thereof to be easily distinguished.

By providing the handle part 42 with the bellows portion 42S, it is possible to easily drain the fluid retained in the handle part 42 from the drain port 45 at a rear end of the handle part 42. Accordingly, any extra effort like the conventional system such that by pushing the fluid against cloth or the like, an amount of fluid in the water-absorbing sponge is adjusted, and a check is performed on whether or not any fibers and/or foreign substances are adhered to the water-absorbing sponge becomes unnecessary. By continuing the drain operation utilizing the bellows function, it is possible to absorb water even after the retaining sponge part 43 absorbs water to lengthen.

Although a case where the retaining sponge part 43 in the surgical sponge 400 has a rectangular parallelepiped having a length of about 6 cm, a width of about 2 cm and a thickness of about 1 cm has been illustrated, the invention is not limited thereto. For example, the retaining sponge part 43 is applicable if it has a dimension and shape such that it extends to an inner wall of the handle part 42 to be not locked when swollen. The retaining sponge part 43 is not limited to one water-absorbing material (illustrated one) having the same length as that of the handle part 42 having the length L2 of 6 cm. Similar to the first embodiment, the retaining sponge part 43 may be selected to have a length (of about 2 cm) divided by three of the handle part 42 and these three parts may be loaded into the handle part 42.

Further, the handle part 12 containing the retaining sponge part 13 as shown in FIG. 1 may be loaded into the bellows handle part 42. When a straw like handle part 12 is loaded in the handle part 42, the handle part 42 pushes down the handle part 12 indirectly so that it is difficult for the absorbed fluid to retain in a drain or the like of the bellows portion 42S.

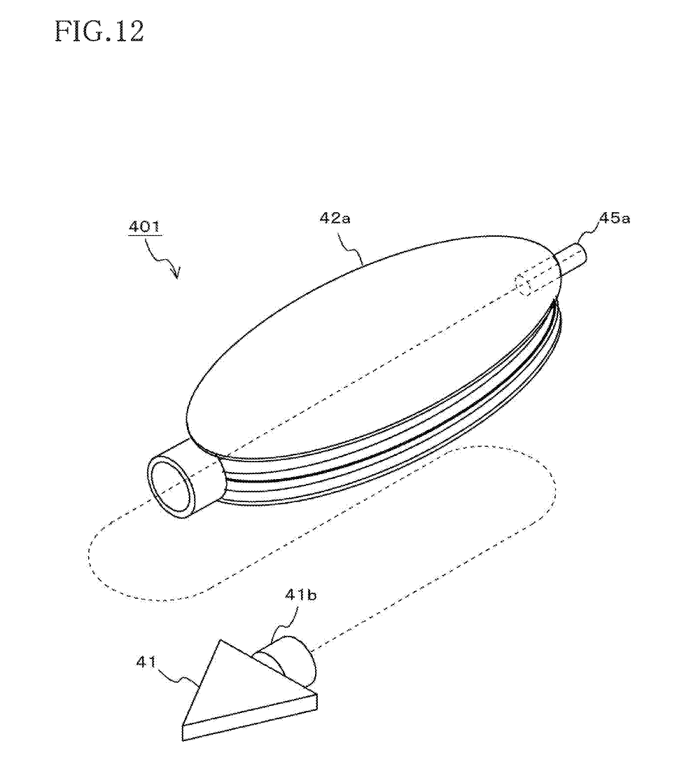

The following will describe configuration examples of surgical sponges 401 through 403 as variations with reference to FIGS. 12 through 14. The surgical sponges 401 through 403 have handle parts 42a, 42b and 42c, which have different shapes from that of the bellows handle part 42 of the surgical sponge 400. The function other than the handle parts 42a, 42b and 42c is identical to the above-mentioned ones, their description will be omitted.

In the surgical sponge 401 shown in FIG. 12, the handle part 42a is a columnar body having an almost oblique bottom surface. In the handle part 42a, the retaining sponge having the almost same shape and dimension as those of the handle part 42a is contained. The fluid absorbed from the tip sponge part 41 is drained from a drain port 45a via the attaching portion 41b and the retaining sponge, not shown, in the handle part 42a.

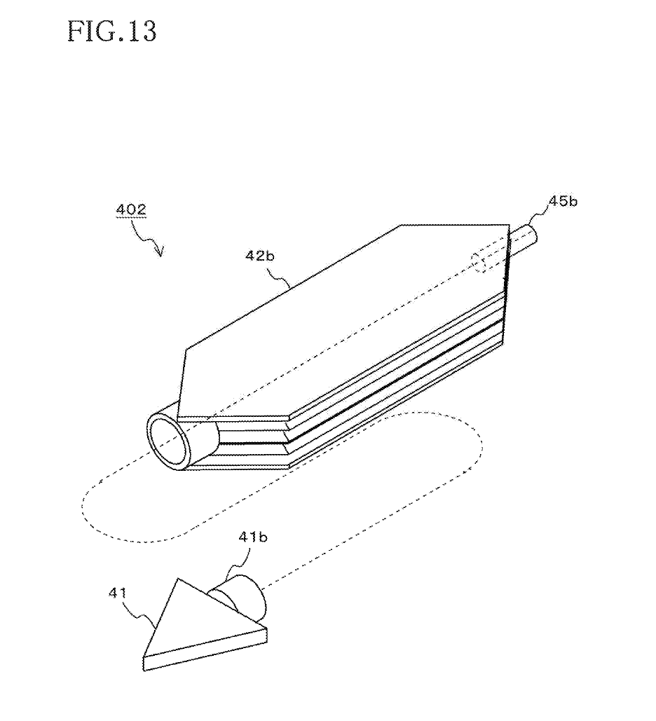

In the surgical sponge 402 shown in FIG. 13, the handle part 42b is a column having a hexagonal bottom surface. In the handle part 42b, the retaining sponge having the almost same shape and dimension as those of the handle part 42b is contained. The fluid absorbed from the tip sponge part 41 is drained from a drain port 45b via the attaching portion 41b and the retaining sponge, not shown, in the handle part 42b. The bottom surface shape of the handle part 42b may be any polygon other than hexagon if it is a shape such that a nursing assistant or the like is easy to hold the same.

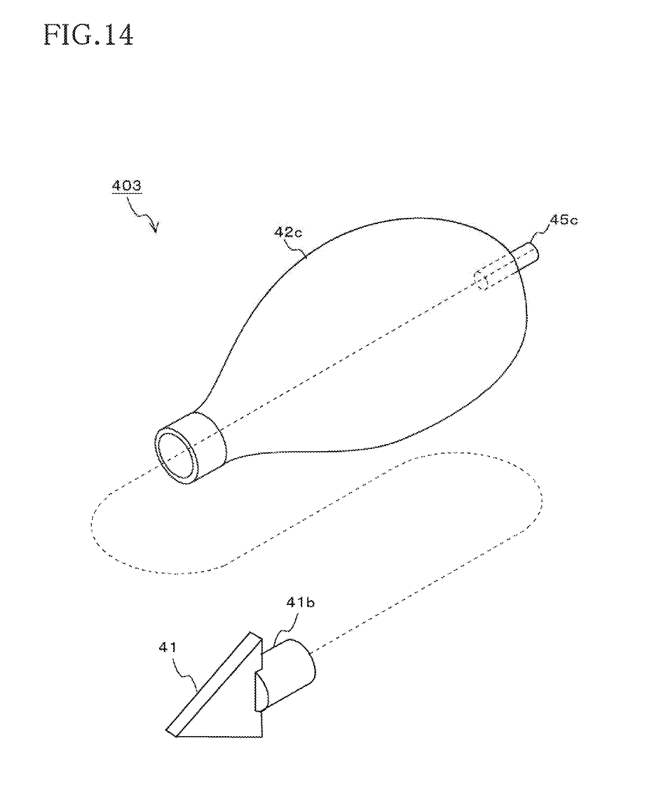

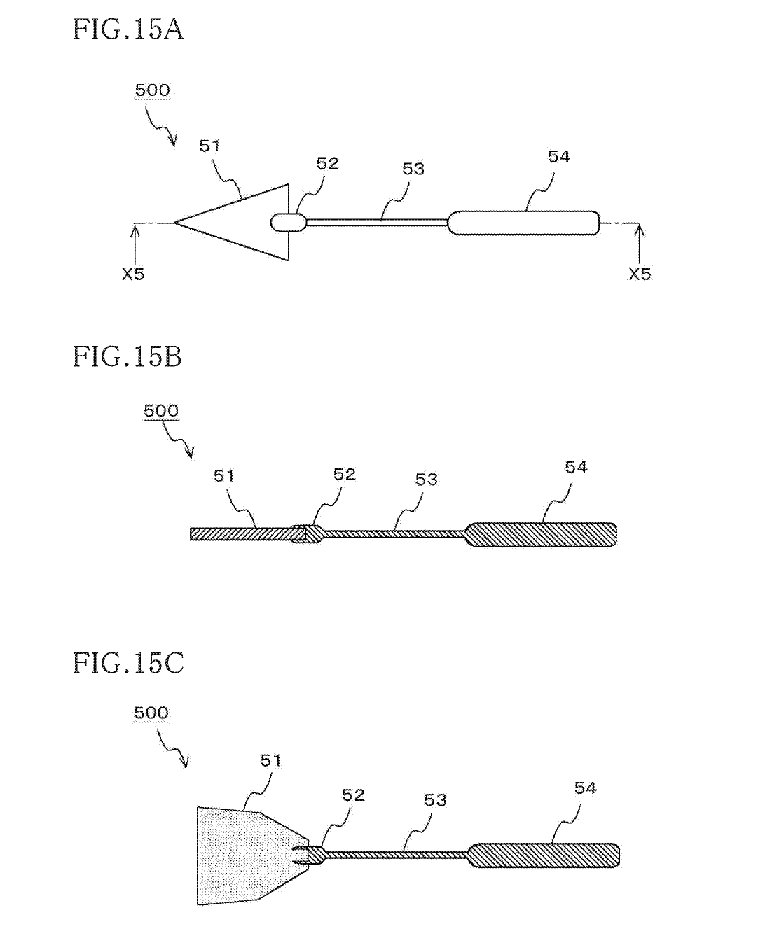

In the surgical sponge 403 shown in FIG. 14, the handle part 42c has a fig like shape. In the handle part 42c, the retaining sponge having the almost same shape and dimension as those of the handle part 42c is contained. The fluid absorbed from the tip sponge part 41 is drained from a drain port 45c via the attaching portion 41b and the retaining sponge, not shown, in the handle part 42c.

Each of the retaining sponges in the surgical sponges 401 through 403 is contained in each of the handle parts without drying and compressing them when producing them, which is different from the retaining sponge part 43 of the surgical sponge 400. Therefore, any workload as to the drying and compressing at the production thereof may be reduced.

Thus, according to the surgical sponge 400 as the second embodiment, it is configured that the retaining sponge part 43 connected to the tip sponge part 41 is loaded into the bellows handle part 42 to absorb the fluid.

According to this configuration, since the fluid absorbed from the tip sponge part 41 can be stored in the handle part 42 and drained from the drain port 45, it can absorb the fluid without remaining. As compared with the first embodiment, an amount of fluid absorbed by the retaining sponge part 43 is increased.

According to the above-mentioned surgical sponge 400, since a water-retaining condition of the retaining sponge part 43 inside the handle part 42 is seen from the outside thereof, it is possible to perform washing (water-retaining) operation while checking the water-retaining condition thereof. It is also possible to drain the fluid stored in the handle part 42 to the outside by utilizing the bellows structure.

According to this surgical sponge 400, the fluid absorbed from the tip sponge part 41 and retained in the retaining sponge part 43 is disposed to the outside by squeezing the bellows portion 42S from the outside from a side of the tip sponge part 41 toward the drain port 45 at rear end thereof. The bellows portion 42S returns to an original shape thereof when receiving no pushing force from the outside so that the retaining sponge part 43 utilizing the capillary phenomenon can be used time after time. The handle part 42 can be used as a pump. This enables to be provided the surgical sponge 400 which can absorb a large amount of the surgical fluid or the like and can be repeatedly used.

Although a case where the tip sponge part 41 is previously attached to the retaining sponge part 43 and these assemblies are loaded into the handle part 42 has been described in the above embodiment, the loading and attaching order of the tip sponge part 41 and the retaining sponge part 43 may alter. For example, a method such that after the tip sponge part 41 is previously attached to the handle part 42, the retaining sponge part 43 is loaded into the handle part 42 so that it is connected to the tip sponge part 41 may be adapted. It is also possible to use the handle part 42 according to this invention in place of the sickle-shaped portion of the conventional apparatus which absorbs water (see the above-mentioned US patent).

Further, when the syringe 90 is connected to the drain port 45 at a terminal end of the handle part 42 through the hose 91 shown in FIG. 6B and a nursing assistant or the like performs the absorption operation, it is possible to drain the fluid absorbed by the surgical sponge 400 to another place. The drainage device is not limited to the syringe 90: A pipette, a suction tube, an electric pump and the like may be used. This allows a fluid delivery system to be built up.

By using the above-mentioned device, it is possible to use the surgical sponge 400 without changing the same even in an absorption operation in a case where a wound of a patient is profound. Further, backflow after the water absorption is limited so that a drainage path can be controlled. This allows the surgical sponge 400 to absorb a larger amount of the fluid.

Although a case where the tip sponge part 41 is connected to the retaining sponge part 43 has been described in this embodiment, this invention is not limited thereto: Any of the tip sponge parts 21, 31 shown in FIGS. 7B and 8B may be attached to the retaining sponge part 43. When the tip sponge part 21 is attached, it is possible to wash a narrow relevant wound. When the tip sponge part 31 is attached, it is possible to perform the washing (water-retaining) operation while looking at a state of the relevant wound through the window 14 because the window 14 passing through the water-absorbing sponge is provided.

It is to be noted that as to the tip sponge parts 11, 41, they may be hardened sponge material which maintains a certain degree of hardness, if they are sponge material having any water-absorption quality, even when they are not rich in flexibility such that the whole of each of them is softened after they absorb the water. Any sweeping effect such as removal of foreign substances in addition to the water-absorption quality can be also obtained.

Further, when the handle part 42 is treated as an appendage of the handle part 12, the handle part 42 which is provided with a check valve, not shown, at the open end of the handle part 42 may be used. For example, there is a case where the handle part 42 is connected to the drain port 15 of the straw-type handle part 12 of the first embodiment to function as a pump. In this case, it is configured that the check valve provided at the open end of the handle part 42 blocks any backflow of the fluid from the handle part 42 to the handle part 12. The surgical sponge 400 or the like thus configured is preferably available for the absorption operation in a case where a wound is profound.

### INDUSTRIAL APPLICABILITY

The present invention is very preferably applied to a surgical sponge for medical application, which absorbs blood occurring in an ophthalmologic surgery using a microscope and/or surgical fluid used for washing a relevant part.

### DESCRIPTION OF CODES

11, 41 Tip Sponge Part (First Fluid-Absorbing Part)
12 Handle Part
13, 43 Retaining Sponge Part (Second Fluid-Absorbing Part)
14 Window
15 Drain port
16 Open End
42 Handle Part
90 Syringe
91 Hose
100, 400, 401-403 Surgical Sponge

1. A surgical fluid-absorbing instrument comprising:
a first fluid-absorbing part that absorbs fluid;
a second fluid-absorbing part that absorbs the fluid, the second fluid-absorbing part being provided on a portion continued from the first fluid-absorbing part; and
a retaining member in which the second fluid-absorbing part is loaded, wherein
at least the second fluid-absorbing part is composed of water-absorbing sponge, and
two modes of a state in which the water-absorbing sponge is compressed on one direction and solidified and a state in which the water-absorbing sponge is extended on one direction and swollen are born.

2. The surgical fluid-absorbing instrument according to claim 1, wherein the surgical fluid-absorbing instrument is composed of a water-absorbing sponge in which the first and second fluid-absorbing parts are integrated.

3. The surgical fluid-absorbing instrument according to claim 2, wherein the first fluid-absorbing part has a compressed and solidified shape in which one of the water-absorbing sponge is compressed on one direction; and
the second fluid-absorbing part has a compressed and solidified shape in which the water-absorbing sponge is compressed on a direction that is perpendicular to the compressed direction of the water-absorbing sponge in the first fluid-absorbing part.

4. The surgical fluid-absorbing instrument according to claim 1, wherein the first fluid-absorbing part is composed of a first fluid-absorbing member in which one of the water-absorbing sponge is compressed on one direction; and
the second fluid-absorbing part is composed of a second fluid-absorbing member in which the other water-absorbing sponge is compressed on a direction that is perpendicular to the compressed direction of the water-absorbing sponge in the first fluid-absorbing part.

5. The surgical fluid-absorbing instrument according to claim 4, wherein the first and second fluid-absorbing parts are configured to be connected to each other with a compressed direction of the first fluid-absorbing part and a compressed direction of the second fluid-absorbing part being perpendicular to each other.

6. The surgical fluid-absorbing instrument according to claim 1, wherein the retaining member is composed of visible material through which an internal portion thereof is transparently seen.

7. The surgical fluid-absorbing instrument according to claim 6, wherein the visible material is configured to be a flexible tube having at least any one quality of reversibility and flexibility.

8. The surgical fluid-absorbing instrument according to claim 6, wherein one water-absorbing sponge or more is loaded into the visible material in their compressed and solidified state, with their swelling directions being aligned.

9. The surgical fluid-absorbing instrument according to claim 6, wherein a drain port is provided at a rear end of the visible material.

10. The surgical fluid-absorbing instrument according to claim 9, wherein the visible material has a bellows structure which is expandable to at least a direction that is perpendicular to a longitudinal direction of the visible material.

11. The surgical fluid-absorbing instrument according to claim 9, wherein a long hose is connected to the drain port of the visible material.

12. The surgical fluid-absorbing instrument according to claim 11, wherein a drainage device is connected to a rear end of the hose.

13. The surgical fluid-absorbing instrument according to claim 1, wherein the first fluid-absorbing part is composed of a first fluid-absorbing member in which the isotropic swellable water-absorbing sponge is processed to have a desired shape.

14. The surgical fluid-absorbing instrument according to claim 1, wherein a window is provided in the water-absorbing sponge constituting the first fluid-absorbing part.

15. A method of producing an surgical fluid-absorbing instrument, the method comprising:
a step of processing a water-absorbing sponge to have a desired dimension and forming a tip fluid-absorbing part;
a step of compressing and solidifying a water-absorbing sponge which is continued to the tip fluid-absorbing part and forming a retaining fluid-absorbing part which is able to be swollen to one direction; and
a step of loading the retaining fluid-absorbing part into a tubular retaining member.
